(19) United States
(12) Patent Application Publication
(10) Pub. No.: US 2026/0151620 A1
(43) Pub. Date: Jun. 4, 2026

(54) IMPLANT MEDICAL DEVICES

(71) Applicant: Case Western Reserve University, Cleveland, OH (US)

(72) Inventors: Hoda Amani Hamedani, Cleveland, OH (US); Allison R. Hess-Dunning, Cleveland, OH (US); Jeffrey R. Capadona, Cleveland, OH (US); Christian A. Zorman, Cleveland, OH (US)

(21) Appl. No.: 18/716,791

(22) PCT Filed: Dec. 6, 2022
(86) PCT No.: PCT/US2022/081024

§ 371 Date: Jun. 5, 2024

Related U.S. Application Data
(60) Provisional application No. 63/286,374, filed on Dec. 6, 2021.
(60) Provisional application No. 63/286,384, filed on Dec. 6, 2021.

(51) Int. Cl. A61N 1/05; A61B 5/00; A61B 5/293; A61M 37/00; A61N 1/36

(52) U.S. Cl. CPC A61N 1/0534; A61B 5/293; A61B 5/6847; A61B 5/6868; A61M 37/00; A61N 1/0551; A61N 1/36125; A61B 2562/0285; A61B 2562/046; A61M 2205/0238; A61M 2205/0244; A61M 2205/04; A61M 2207/00; A61M 2210/04

(57) ABSTRACT

An implantable medical device includes a biocompatible flexible substrate and a bioactive agent release coating on at least a portion of a surface of the substrate. The bioactive agent release coating includes an oriented nanotube array of an inorganic material and at least one bioactive agent disposed on and/or in the nanotube array. The nanotube array provides delayed or extended release of a bioactive agent from the coating.

### RELATED APPLICATION

This application claims priority from U.S. Provisional Application Nos. 63/286,374, filed Dec. 6, 2021, and 63/286,384 filed Dec. 6, 2021, the subject matter of which are incorporated herein by reference in their entirety.

### BACKGROUND

The past decade has witnessed significant advances in development of implantable medical devices (IMDs) for biological research and clinical medicine. Despite these advances, IMDs can fail for various reasons, such as poor tissue integration due to mechanical mismatch at the biotic-abiotic interface, inflammation and infection when interfaced with various biological organs and tissues. These challenges continue to call for innovative approaches for the development of multifunctional platforms that enable key functions of the implants while maintaining the tissue's desired biological functions during implantation. In general, biocompatibility, corrosion resistance, anti-inflammatory and antibacterial properties comprise the main requirements of the materials used in all types of medical implants and implantable devices. One major aspect of designing biocompatible platforms with desired functionality is to use materials that closely mimic the mechanics of the living tissue that surrounds the implant. Material-tissue integration may involve important distinctions depending on the types of implant/tissue pertinent to specific applications. For instance, polymeric materials can provide effective biomimetic scaffolds for soft tissues, such as promoting endogenous nerve cell attachment and proliferation, whereas porous inorganic materials are ideal for interfacing with porous structure of hard connective tissue to enhance ingrowth of mineralized tissue in artificial bone/dental implants. Nanostructured materials have provided unique opportunities to design components with multiple functions incorporated into a single structure to optimize the overall performance of the device while minimizing adverse effects associated with inappropriate form factors.

### SUMMARY

Embodiments described herein relate to a medical device and particularly, relate, to an implantable medical device that includes a biocompatible flexible substrate and a bioactive agent release coating on at least a portion of a surface of the substrate. The biocompatible flexible substrate can provide a soft interface material that can be implanted as an implant or coated onto a harder material for an implant. The biocompatible flexible substrate can include viscoelastic properties that match that of an organ or tissue. The bioactive agent release coating can include an oriented nanotube array of an inorganic material and at least one bioactive agent disposed on and/or in the nanotube array. The nanotube array can provide delayed or extended release of a bioactive agent from the coating.

In some embodiments, the implantable medical device can include a transdermal implant with a biocompatible flexible substrate and a bioactive agent release coating that are configured to interface with epithelial tissue. The bioactive agent release coating can include vertically-oriented nanotube arrays of inorganic materials that can provide controlled release of bioactive agents, such as therapeutic agents, over an extended period of time to epithelial tissue. The transdermal implant can be used for a variety of applications in the form of general or stimuli-responsive skin patches for the treatment of chronic and/or non-healing wounds while preventing infections.

In other embodiments, the implantable medical device can include an implantable electrode assembly that includes a biocompatible flexible substrate and bioactive agent releasing microelectrodes that are configured to interface with tissue, such as neural tissue. The bioactive agent releasing microelectrodes can provide electrical stimulation to and electrical recording of neural tissue. The bioactive agent releasing microelectrodes can include inorganic nanotube arrays that are configured for bioactive agent, e.g., anti-inflammatory, antioxidant, growth factor, genes, etc., release with (high spatial precision) with simultaneous electrical recording and stimulation.

In some embodiments, the nanotube arrays geometrical characteristics (e.g., nanotube pore diameter and length) can strongly influence release kinetics, which can be tuned to sustain the release of the bioactive agent over several weeks or months.

In some embodiments, the oriented nanotube array includes a valve metal or valve metal oxide nanotube array or oriented nanotube array of an anodized valve metal, such as a titanium or titania nanotube array.

In some embodiments, the nanotubes of the nanotube array are oriented in substantially the same direction relative to the surface of the substrate. For example, the nanotubes of the nanotube array can be oriented substantially perpendicular to the surface of the substrate.

In some embodiments, nanotubes of the nanotube array can have a pore diameter of about 30 nm to about 100 nm and a length of about 0.5 μm to about 30 μm.

In some embodiments, the nanotubes are annealed and/or reactive ion etched.

In other embodiments, the oriented nanotube array is configured to define at least one electrode on the surface of the substrate. The oriented nanotube array configured as an electrode can have an electrical impedance |Z|<20 Ω·cm² at 1 KHz.

In some embodiments, the nanotubes can include at least one coating, such as a titanium nitride coating.

In some embodiments, the biocompatible flexible substrate can include a compliant biocompatible polymer or textile, such as a polyimide or polyvinyl acetate film or sheet.

In some embodiments, the bioactive agent release coating can provide sustained release of the bioactive agent upon implantation of the implantable medical device in a subject, preferably sustained release over a duration of time of weeks to months upon implantation.

In some embodiments, the oriented nanotube array defines at least one electrode that extends outward from the substrate and is configured to interface with neural tissue of a subject. The at least one electrode can be electrically connected via at least on electrical interconnect to a driving unit and/or recording unit. The driving unit can drive the electrode for electrical stimulating regions of the neural tissue and the recording unit can obtain neural recordings from the electrode.

In some embodiments, the at least one electrode can be part of an electrode assembly. The electrode assembly can include, for example, an intracortical electrode array, an inferior colliculus implant, a deep brain stimulator, an electrocorticography array, a spinal cord electrode, or a brain stem implant.

In some embodiments, the bioactive agent released by the oriented nanotube array that defines the at least one electrode can include at least one of a neuromodulation agent or an anti-inflammatory agent.

Other embodiments described herein relate to a method of forming a bioactive agent releasing implantable medical device. The method can include depositing a valve metal film on a biocompatible flexible substrate. The valve metal film is then electrochemically anodized in an electrolyte at a voltage effective to form an oriented nanotube array. At least one bioactive agent is then loaded in and/or on the oriented nanotube array.

In some embodiments, the method can further include applying a photoresist to select portions of the deposited valve metal film such that the deposited valve metal film includes photoresist covered portions and uncovered portions. The oriented nanotubes only form on uncovered portions of the valve metal film during electrochemical anodization.

In some embodiments, the method further includes removing the photoresist after formation of oriented nanotubes to provide defined regions of valve metal film with oriented nanotube arrays and non-anodized valve metal.

In some embodiments, the oriented nanotube array can be etched or machined to provide defined regions of valve metal film with and without oriented nanotube arrays.

In some embodiments, the valve metal film includes a titanium metal film and the oriented nanotube array comprises a titania nanotube array.

In some embodiments, the electrolyte includes an aqueous mixture of ammonium fluoride and ethylene glycol.

In some embodiments, the oriented nanotube array can be annealed and optionally etched.

In other embodiments, the method can include providing a coating on at least a portion of oriented nanotube array. The coating can include, for example, a titanium nitride coating.

Still other embodiments relate to a method of forming a bioactive agent releasing implantable electrode. The method can include electrochemically anodizing a first surface of a titanium foil in an electrolyte at a voltage effective to form an oriented titania nanotube array on the first surface of the foil. An opposite second surface of the titanium foil can be adhered to a biocompatible flexible substrate. A photoresist can then be deposited and patterned on the first surface of the titanium foil. The photoresist covered titanium foil can be etched to form a plurality of titania nanotube array microelectrodes on the flexible polymer substrate and any remaining photoresist can be removed after etching. At least one bioactive agent can then be loaded in and/or on the oriented nanotube array.

### BRIEF DESCRIPTION OF THE DRAWINGS

FIG. 1(A-B) illustrate an overview of drug release mechanisms. (A) Rapid drug release from hydrogels and polymeric nanoparticles produces a burst effect that results in a spike in drug concentration, while the constrained diffusion characteristic of nanotubes leads to a steady and sustained linear release. (B) Zero-order diffusion produces stable drug concentrations in vivo.

FIG. 2(A-B) illustrate a (A) schematic diagram of electrochemical anodization set-up and (B) TiO₂ nanotube formation mechanism on Ti-sputtered polyimide substrate shown in the photograph (top). The photograph of anodized Ti-sputtered Kapton (Bottom) reveals a transparent film.

FIG. 3(A-F) illustrate SEM cross-section (top row) and top-surface (bottom row) images of titania nanotube arrays with various tube lengths and pore sizes that were grown on Ti foil at (A, D) 40 V, (B, E) 20 V and (C, F) 10V.

FIG. 4 illustrates sustained linear DEX release from a ˜500 um-deep, 85 nm-diameter TNAs on polyimide substrate for more than 6 weeks (43 days). Insets: Cross section views SEM images of a free-standing TNAs grown on polyimide substrate.

FIG. 5(A-C) illustrate direct patterning of TNAs on polyimide substrate using picosecond laser micromachining.

FIG. 6(A-B) illustrate (A) Patterning and transferring TNA films to polymer substrates. (B) Release profile of Resveratrol from TNA (2 micron long, 30 nm pore size) at 37° C.

FIG. 7 illustrates a schematic showing further process of the TNA.

FIG. 8 illustrates normalized impedance values at three frequencies for non-anodized titanium foil samples and anodized, annealed, and nitrogen-doped TNA samples. The gray box indicates the targeted impedance at 1 KHz.

FIG. 9 illustrates representative impedance spectra for Ti and TNA samples.

FIG. 10(A-B) (A) illustrate SEM images of annealed TNA samples before and after reactive ion etching in a CF₄/O₂ plasma. Scale bar=1 μm. (B) During the RIE step, a spongy oxide surface layer is removed.

FIG. 11 illustrates a comparison of the 1 kHz electrochemical impedance magnitude of annealed TNA samples before and after RIE etching in a CF₄/O₂ plasma.

Regardless of initial impedance, the final impedance measurements were all similar.

FIG. 12 illustrates representative electrochemical impedance spectroscopy (EIS) plots before and after etching for 60 seconds.

FIG. 13 illustrates a schematic of a flexible electrode assembly in accordance with an embodiment.

FIG. 14 illustrates a schematic showing fabrication of a device in accordance with one embodiment.

FIG. 15(A-E) illustrate (A) TNA microsegments patterned on PDMS film. (B) Completed TNA-NC neural probe connected to printed circuit board. (C) Individual TNA microelectrode integrated into TNA-NC neural probe with Au trace. (D) TNA-NC neural probe wrapped around tweezers to demonstrate mechanical flexibility. (E) TNA-NC neural probe inserted into agarose gel demonstrates functional robustness of the TNAs integrated onto the mechanically-softening substrate with interconnecting traces. Scale bar=200 μm.

### DETAILED DESCRIPTION

Unless otherwise defined, all terms (including technical and scientific terms) used herein have the same meaning as commonly understood by one of ordinary skill in the art to which this invention belongs. Accordingly, these terms can include equivalent terms that are created after such time. It will be further understood that terms, such as those defined in commonly used dictionaries, should be interpreted as having a meaning that is consistent with their meaning in the present specification and in the context of the relevant art, and will not be interpreted in an idealized or overly formal sense unless expressly so defined herein.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

It will be understood that when an element, such as a layer, region or substrate, is referred to as being “on,” connected to” or “coupled to” another element, it can be directly on, connected or coupled to the other element, or one or more intervening elements may be present. In contrast, when an element is referred to as being “directly on,” “directly connected to” or “directly coupled to” another element, there are no intervening elements present.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Furthermore, relative terms, such as “lower” or “bottom” and “upper” or “top,” may be used herein to describe one element's relationship to another element as illustrated in the figures. It will be understood that relative terms are intended to encompass different orientations of the device in addition to the orientation depicted in the figures. For example, if the device in the figures is turned over, elements described as being on the “lower” side of other elements would then be oriented on “upper” sides of the other elements. The exemplary term “lower”, can therefore, encompasses both an orientation of “lower” and “upper,” depending on the particular orientation of the figure.

As used herein, the terms “implant”, “implant device”, “implantable device”, and “implantable medical device” refer to any device that may be inserted into the body of a patient. Implant devices may be used to treat a patient for any reason. In some embodiments, an implant device is used to treat one or more disorders.

The term “bioactive agent” can refer to any agent capable of affecting cell or tissue activity, function, growth, formation, destruction, and/or targeting. Examples of bioactive agents can include, but are not limited to, pharmaceuticals, vitamins, chemotactic agents, various proteins (e.g., short term peptides, bone morphogenic proteins, collagen, glycoproteins, and lipoprotein), cell attachment mediators, biologically active ligands, integrin binding sequence, various growth and/or differentiation agents and fragments thereof (small molecules that affect the upregulation of specific growth factors, tenascin-C, hyaluronic acid, chondroitin sulfate, fibronectin, decorin, thromboelastin, thrombin-derived peptides, heparin-binding domains, heparin, heparan sulfate, polynucleotides, DNA fragments, DNA plasmids, MMPs, TIMPs, interfering RNA molecules, such as siRNAs, oligonucleotides, proteoglycans, glycoproteins, glycosaminoglycans, and DNA encoding for shRNA.

The terms “biodegradable” and “bioresorbable” may be used interchangeably and refer to the ability of a material (e.g., a polymer or textile) to be fully resorbed in vivo. “Full” can mean that no significant extracellular fragments remain. The resorption process can involve elimination of the original implant material(s) through the action of body fluids, enzymes, cells, and the like.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “layer” denotes any arrangement whereby one portion of the material has a different chemical composition than another. The layer may include any number of individual layers, and the interface between the layer(s) may be sharp or gradual. For example, a nanotube oxide layer on the surface of an implant having a metal-containing surface may denote a layer deposited on the surface, or may denote a layer formed below the surface by oxidation of the metal on the surface of the implant. The interface between the oxide layer and the underlying implant may be sharp or gradual. For example, the interface between the oxide layer and the underlying implant may comprise a gradual rise in the amount of oxide present from, say 0% in the underlying implant to 50% in the oxide layer, over a certain thickness of the implant.

Embodiments described herein relate to a medical device and, particularly, relate to an implantable medical device that includes a biocompatible flexible substrate and a bioactive agent release coating on at least a portion of a surface of the substrate. The biocompatible flexible substrate can provide a soft interface material that can be implanted as an implant or coated onto a harder material for an implant. The biocompatible flexible substrate can include viscoelastic properties that match that of an organ or tissue. The bioactive agent release coating can include an oriented nanotube array of an inorganic material and at least one bioactive agent disposed on and/or in the nanotube array. The nanotube array can provide delayed or extended release of the bioactive agent from the coating.

The biocompatible flexible substrate can include any flexible polymer or textile material that is biocompatible and compatible with the mechanical properties of tissue on and/or in which the device is implanted. The biocompatible flexible polymer material can also have selective electrical conductivity to enable the transmission of electrical signals to and from tissue or cells, such as neurons.

In some embodiments, the biocompatible flexible substrate can be mechanically flexible and stretchable, so that it can move and flex with the tissue as it moves or grows. For example, the biocompatible flexible substrate can move and flex with a nerve as the nerve moves and grows, minimizing the forces that the substrate applies to the nerve cells.

In some embodiments, the biocompatible flexible substrate can be made of a biocompatible polymer base material that will interact in a safe and healthy manner with surrounding cells and tissue. For example, the base polymer can comprise a polysiloxane, such as polydimethylsiloxane (PDMS); an unsaturated polyester, such as polybutylene fumarate (PBF) or polypropylene fumarate (PPF), as well as copolymers thereof (e.g., PPF/poly(propylene fumarate diacrylate) (PPF/PPF-DA)); a polyolefin, such as poly(butadiene), polybutadiene and polynorbornene copolymers; a polyurethane (PU), such as PU copolymers including PU-polycaprolactone (PL-PCL), poly(ester urethane)urea (PEUU), poly(ether ester urethane)urea (PEEUU), and thermoplastic polyurethane (TPU), such as polyester-based TPUs (mainly derived from adipic acid esters) or polyether-based TPUs (mainly based on tetrahydrofuran ethers); a polyimide; a polyethylene glycol (PEG) (or poly(ethylene oxide) (PEO), including poly(ethylene glycol fumarate) (PEGF), oligo(PEG fumarate) (OPF, a hydrogel), as well as PEG copolymers such as di-and tri-block polymers polycaprolactone-polylactide-PEG/PEO (PCL-PLA-PEO/PEG), PCL-PEO-PCL, PCL-PEG, PLA-PEG, those including poly[(lactic acid)-co-(glycolic acid)-alt-(γ-benzyl-L-glutamic acid)] (PLGBG) such as PLGBG-PEG-PLGBG, those including poly [(lactic acid)-co-[(glycolic acid)-alt-(L-glutamic acid)] (PLGG) such as PLGG-PEG-PLGG, PEG-PCL-PEG, PEG/poly(DTE carbonate), ethylene-vinylacetate copolymer, and poly(ether ester amide)s (PEEAs) formed by polycondensation of PEG and diester-diamide to create an amphiphilic system); or a ring opening metathesis polymerization (ROMP) formed polymer, such as norbornene. It can have patterned holes, channels, or openings through which tissue or cells, such as neurons, can grow.

Further, the biocompatible flexible substrate can be made electrically conductive by adding some volume fraction of conductive particles (e.g., carbon nanotubes and other particles) and/or conductive particle precursors (e.g., graphene oxide or a metal salt which can be thermally reduced, such as with silver nitrate) to the polymer base material. As the volume fraction of particulate/precursor is increased in a composite material, the composite material's physical properties transition from being approximately equal to the properties of the polymer base material to approaching the properties of the particulate/precursor. Therefore, as the volume fraction of electrically conductive particles/precursors is increased, the electrical conductivity of the composite material increases accordingly.

By selecting particles/precursors with large aspect ratios (ratio of length to diameter), such as carbon nanotubes, the electrical properties of the composite can be made to transition to the properties of the particles at a lower volume fraction than the mechanical properties. Therefore, polymer composites can be created with high electrical conductivity (approaching the conductivity of carbon nanotubes) and with low elastic modulus and high yield strain (approaching the mechanical properties of the base polymer). The interface can be made to be selectively conductive by locally varying the concentration of conducting particulates. That is, specific regions of the substrate proximate the electrode sites can be made electrically conductive by filling with a high local volume fraction of conducting particulates, while other regions between the electrode sites can be made to be electrical insulators by using a low (or zero) local volume fraction of conducting particulates.

The oriented nanotube array can be attached to or formed on the biocompatible flexible substrate by anodizing and attaching a metal foil to the biocompatible flexible substrate or anodizing metal film deposited on the biocompatible flexible substrate.

In some embodiments, the oriented nanotube array includes a valve metal or valve metal oxide nanotube array or oriented nanotube array of an anodized valve metal. The valve metal can be selected from aluminum, titanium, tantalum, niobium, tungsten, chromium and/or one or more metal alloys such as titanium alloys (e.g., Ti₆Al₄V, Ti₆Al₄V_{0.5}Pt, Ti₆Al₇B, Ti₆Al₇Nb_{0.5}Pt, Ti₅Al_{0.5}B, Ti₅Al_{2.5}Fe, Ti_{4.2}Fe_{6.9}Cr, Ti_{4.2}Fe_{6.7}Cr₃Al, Ti₁₅Mo₅Zr₃Al, Ti₁₅Mo₃Nb₃0, Ti₁₂Mo₆Zr₂Fe, Ti₃₅B₇Zr₅Nb, Ti₃₅Nb₇Zr₅Ta, Ti₃₅Nb₇Zr₅Ta_{0.}4O, Ti₂₉Nbi₃Ta₇Zr, Ti₂₉Nb₁₃Ta₂Sn, Ti₂₉Nb₁₃Ta_{4.5}Zr, Ti₂₉Nb₁₃Ta_{4.6}Sn, Ti₂₉Nb₁₃Ta₆Sn, Ti₂₉Nb₁₃Ta₄Mo, T₂₉Nbi₃Ta_{4.6}Zr, Ti₁₆Nbi₃TaMo, Tii₃Nbi₃Zr, Ti_{0.5}Pt, titanium-molybdenum alloys and titanium-tantalum alloys).

In some embodiments, the nanotubes of the nanotube array are oriented in substantially the same direction relative to the surface of the substrate. For example, the nanotubes of the nanotube array can be oriented substantially perpendicular to the surface of the substrate.

The nanotubes of the oriented nanotube array can have an average and/or mean pore diameter in the range of about 1 to about 200 nm (e.g., about 5 nm, about 10 nm, about 20 nm, about 30 nm, about 40 nm, about 50 nm, about 60 nm, about 70 nm, about 80 nm, about 90 nm, about 100 nm, about 125 nm, about 150 nm, about 200 nm). In some embodiments, the nanotubes can have an average and/or mean pore diameter of at least about 1 to about 200 nm (at least about 5 nm, at least about 10 nm, at least about 20 nm, at least about 30 nm, at least about 40 nm, at least about 50 nm, at least about 60 nm, at least about 70 nm, at least about 80 nm, at least about 90 nm, at least about 100 nm, at least about 125 nm, at least about 150 nm, at least about 200 nm). In some embodiments, the nanotubes can have an average and/or mean pore diameter of less than about 1 to about 200 nm (less than about 5 nm, less than about 10 nm, less than about 20 nm, less than about 30 nm, less than about 40 nm, less than about 50 nm, less than about 60 nm, less than about 70 nm, less than about 80 nm, less than about 90 nm, less than about 100 nm, less than about 125 nm, less than about 150 nm, less than about 200 nm).

In some embodiments, the nanotubes can have an average and/or mean height in the range of about 0.5 μm to about 30 μm (e.g., about 0.5 μm, about 1 μm, about 2 μm, about 3 μm, about 4 μm, about 5 μm, about 6 μm, about 7 μm, about 8 μm, about 9 μm, about 10 μm, about 12 μm, about 15 μm, or about 20 μm, about 22.5 μm, about 25 μm, about 27.5 μm, about 30 μm). In some embodiments, the nanotubes can have an average and/or mean height of at least about 1 μm to about 30 μm (at least about 0.5 μm, at least about 1 μm, at least about 2 μm, at least about 3 μm, at least about 4 μm, at least about 5 μm, at least about 6 μm, at least about 7 μm, at least about 8 μm, at least about 9 μm, at least about 10 μm, at least about 12.5 μm, at least about 15 μm, at least about 20 μm, at least about 22.5 μm, at least about 25 μm, at least about 27.5 μm, at least about 30 μm). In some embodiments, the nanotubes have an average and/or mean height of less than about 0.5 μm to about 30 μm (less than about 0.5 μm, less than about 1 μm, less than about 2 μm, less than about 3 μm, less than about 4 μm, less than about 5 μm, less than about 6 μm, less than about 7 μm, less than about 8 μm, less than about 9 μm, less than about 10 μm, less than about 12.5 μm, less than about 15 μm, less than about 20 μm, less than about 22.5 μm, less than about 25 μm, less than about 27.5 μm, less than about 30 μm).

The oriented nanotube array can be formed by electrochemical anodization of the surface of a metal foil or film. Metal foil or film can be attached to or deposited on the flexible biocompatible substrate or be separate from the flexible biocompatible substrate during anodization. In general, the surface of the metal foil or film functions as the anode during the electrochemical anodization process. Oxidation of the metal surface of the anode occurs and given appropriate reaction conditions, nanotubes are formed on the surface of the foil or film.

The surface of the foil or film may be prepared before the electrochemical anodization process is performed. For example, the surface may be cleaned using distilled water and isopropyl alcohol or methyl ethyl ketone washes, optionally combined with ultrasonic agitation of the washes to further help remove impurities from the surface of the foil or film. The surface also may be cleaned by a chemical-mechanical polishing process or simply a mechanical polishing process, for example, using a diamond paste.

In one example, titanium foil of varying thicknesses, such as 0.25, 0.5 μm, 1.0 μm, 10 μm, 20 μm, 30 μm, 40 μm, 50 μm, 60 μm, 70 μm, 80 μm, 90 μm, 100 μm, 200 μm, 500 μm, 1 mm, or 2.0 mm thick samples, can be cleansed with acetone followed by an isopropyl alcohol rinse before anodization. Although specific thicknesses are referenced it should be appreciated that the foil can be of any thickness amenable to anodization. The thickness for formed oxide is a function of thickness for the working electrode, such as for example the thickness of the titanium foil/film. The titanium foils constitute “thick films” as is commonly appreciated and known by skilled artisans. The titanium foils can have a sufficient thickness to provide enough rigidity and stability to be handled and to facilitate anodization.

The electrochemical anodization process may occur in a suitable electrolyte solution. In the case of titanium and titanium alloy-containing foils or films, the anodization can be performed in a two-electrode configuration with the titanium foil of film as the working electrode and platinum foil as the counter electrode, under constant potential at room temperature, approximately 22° C. Although anodization can be performed at room temperature, it should be appreciated that anodization could occur over a variety of temperatures. For example, anodization could be performed from −5° C. to 100° C. or any other temperature range amenable to anodization for forming the nanotube array of varying geometries and morphology.

An electrolytic bath can be used to anodize titanium foil providing synthesis of self-aligned self-standing nanotube arrays of, for example, 0.5 μm to 30 μm in length. The nanotube array can have any packing arrangement, such as hexagonal packing arrangement, but alternate nanotube packing arrangements can be used for the titania nanotube array. However, as compared to other perceivable packing arrangements, the hexagonal arrangement provides superior structural integrity of the array and best closes the gaps between adjacent tubes within the nanotube array. Limiting the gap between adjacent tubes in the array limits unwanted materials from entering and introducing imperfections into the array. Those skilled in the art can appreciate that the electrolyte may be an aqueous solution, such as an amide based electrolyte, or a non-aqueous electrolyte, such as a polar organic electrolyte. The time-dependent anodization current may be recorded using a computer controlled multimeter and the as-anodized samples ultrasonically cleansed in deionized water to remove surface debris. The morphology of the anodized samples can be studied using a field emission scanning electron microscope (FESEM).

As described in the Example, ethylene glycol can be used as a solvent in electrochemical oxidation and exhibits an extremely rapid titania nanotube growth rate (e.g., up to 15 μm/min). The nanotubes formed in ethylene glycol exhibited long range order manifested in hexagonal close-packing and very high aspect ratios. Ethylene glycol was also found to minimize lateral etching of the nanotube array. As such, the nanotube array can exhibit substantially uniform wall and pore thicknesses, unlike the as-anodized nanotubes anodized in other aqueous electrolytes that dissolve the walls and pores of the tube more at the top of the sample than at the bottom due to the top-up formation of the tube (i.e., the top portion of the nanotube is in the electrolyte solution longer and is exposed to the dissolving effects of the electrolyte for longer than the bottom portion). Although ethylene glycol is highly amenable to electrochemical oxidation, it should be appreciated that the electrolyte is not limited to those containing solely ethylene glycol, and other polar organic electrolytes, such as formamide (FA), dimethyl sulfoxide (DMSO), Dimethylformamide (DMF), and N-methylformamide (NMF) can also be used.

In some embodiments, the electrolyte for forming vertically oriented TiO₂ nanotube arrays can further include hydrofluoric acid (HF), potassium fluoride (KF), or ammonium fluoride (NH₄F). Using electrolytes having sufficient fluoride ions, such as NH₄F, can provide adequate etching of the TiO2. For example, nanotube array can be obtained using an ethylene glycol electrolyte containing a sufficient wt % NH₄F and H₂O.

Generally, the specific electrolyte solution used will depend upon the composition of the metal foil or film to be anodized. Therefore, an electrolyte solution useful for the formation of nanotubes on the surface of a titanium foil or film may be different, for example, from an electrolyte solution useful for the formation of nanotubes on the surface of a tantalum foil or film. One skilled in the art will appreciate other electrolyte solutions that successfully may be used in the anodization process to create nanotubes on the surface of the implant.

It may be preferable to choose as a cathode, an inert, corrosion resistant metal. For example, gold, iridium, platinum, rhodium, palladium, and ruthenium are among the metals contemplated for use as the cathode in the electrochemical anodization process. One skilled in the art will appreciate other materials may be used as the cathode in the electrochemical anodization process.

During electrochemical anodization, an electrical potential is applied between the anode and the cathode placed in the electrolyte solution by an outside electrical source. The electrical potential may vary from about 1 V to about 60 V, preferably from about 5 V to about 40 V, and most preferably from about 10 V to about 40 V. In some embodiments, where the implant has a titanium or titanium alloy-containing surface and the concentration of NH₄F in the electrolyte solution is about 0.5% by weight, the electrical potential may be anywhere from about 10 V to about 40 V. The electrical potential for modification of the surface of the foil or film may be dependent upon the concentration of the acid or base in the electrolyte solution. Generally, higher voltages may be needed to produce the desired nanotube arrays when more dilute electrolyte solutions are used.

Additionally, the electrical potential may affect physical properties of the nanotubes formed on or in the surface of the foil or film. In general, higher voltage potentials may yield nanotubes with larger pore diameters. Therefore, by choosing the appropriate voltage, nanotubes with a desired pore diameter may be formed on or in the surface of the foil or film. Also, the electrical potential may be varied during the electrochemical anodization process, resulting in the formation of nanotubes with a tapered structure. A tapered nanotube structure with a large base and narrow top may be desirable, for example, in order to create reservoirs to trap bioactive agents and additives onto or in the nanotube array on the surface of the foil or film, particularly in the case of drug depots.

The nanotubes of the nanotube arrays formed by the electrochemical anodization process are typically oxides of the metal foil or film. For example, in the case of a titanium foil or film, titanium oxide (TiO₂) nanotubes are formed on the surface, in the case of a foil or film having a Ti—Al—V titanium alloy-containing surface, the nanotubes may comprise titanium oxide (TiO₂), aluminum oxide (Al₂O3), and vanadium oxide (VaO₂). In the case of a foil or film having an aluminum-containing surface, aluminum oxide (Al₂O3) nanotubes may be formed.

Generally, the oxide nanotubes also may incorporate elements from the electrolyte solution in which the electrochemical anodization process takes place. For example, nanotubes formed on the surface of titanium and titanium alloy-may incorporate small amounts of fluorine in their structure because ammonium fluoride may be used in the electrolyte solution for electrochemical-anodization of titanium and titanium alloy-containing implants. It may be advantageous to mix certain additives into the electrolyte solution in anticipation of the additives being incorporated into the nanotubes formed on the surface of the foil or film. For example, ionic substances may be mixed into the electrolyte solution so that the ionic substances will be incorporated into the nanotubes formed on the surface of the foil or film. An ionic component in the oxide nanotubes may be advantageous in order to increase the nanotubes'ability to retain beneficial bioactive agents and additives that are to be adsorbed onto or incorporated into the nanotubes before, during, or after implantation of the device.

The size of the nanotubes is one property of the nanotubes that may be adjusted by varying process variables such as voltage, time, and composition of the electrolyte solution. It is believed that the electrochemical anodization of a titanium or titanium alloy-containing foil or film may yield nanotubes with an inner diameter between about 15 nanometers and about 200 nanometers, an outer pore diameter between about 15 nanometers and about 300 nanometers, and a height between about 0.5 μm and about 30 μm. However, it also is contemplated that optimization of the electrochemical anodization process as applied to titanium and titanium alloy-containing foil or film may yield nanotubes with dimensions outside of these ranges. Additionally, it is contemplated that nanotubes formed from other metals and alloys may be produced in different ranges of sizes, dependent upon the metal or alloy that comprises at least the surface of the foil or film.

For example, it is thought that higher voltage potentials may yield nanotubes with larger pore diameters. Therefore, by choosing an appropriate voltage, nanotubes with a desired pore diameter may be formed. In order to vary the shape of the nanotubes, for example, the electrical potential may be varied during the electrochemical anodization process. This may result in the formation of tapered nanotubes or otherwise irregularly shaped nanotubes. The height and pore diameter of the nanotubes also may be influenced by the composition of the electrolyte solution. For example, a more dilute electrolyte composition may delay nanotube formation, thereby decreasing the height of the nanotubes produced over a given time period compared with a more concentrated electrolyte solution.

Also, the duration of time during which the foils or films are modified maybe adjusted to attain desired nanotube structures. For example, increasing the duration of the modification process may result in the creation of nanotubes of increased height and more developed structure.

It has been observed, in relation to titanium and titanium alloy-containing foils or films, that the proper execution of the electrochemical anodization process to form oxide nanotubes on the surface of the foil or film may result in a three-part structure. On the immediate surface of the foil or film are the oxide nanotubes, aligned generally perpendicular to the surface geometry of the foil or film. Below the oxide nanotubes is the interface between the nanotube layer and the titanium surface. The interface may also comprise an oxide of the titanium or titanium alloy. Below the interface between the nanotube layer and the titanium surface is the titanium itself. Without desiring to be limited to any theory of operation, it is believed that similar structures may be observed in the surface electrochemically anodized foils or films comprising other metals and metal alloy surfaces.

Following electrochemical anodization and formation of nanotubes on the surface of the foil or film, the nanotube array can be further processed by, for example, etching or machining the nanotubes to provide defined regions of metal foil or film with and without oriented nanotube arrays.

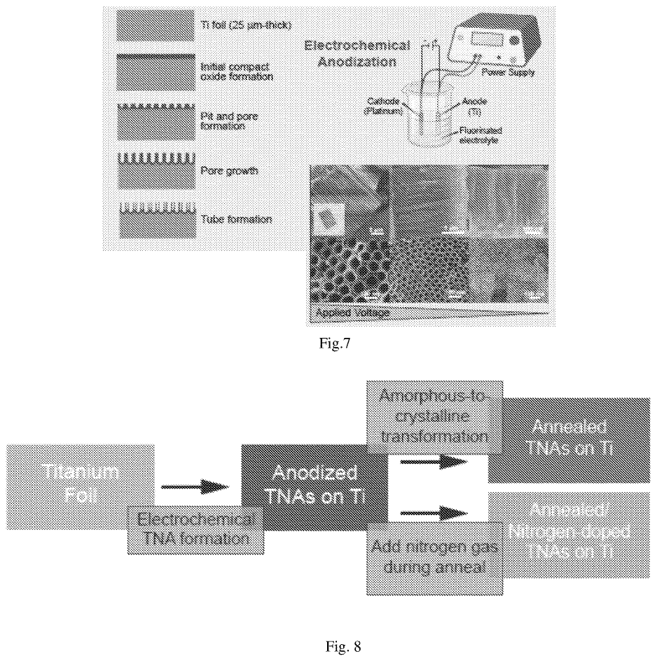

The nanotube array can also undergo further treatment to impart advantageous properties to the nanotubes or device. For example, as shown in FIG. 7 the nanotube array may be annealed to modify the crystalline structure of the nanotubes and enhance the electrochemical impedance of the nanotubes when used as an implantable electrode for neuromodulation and neural recording applications. For example, the titanium oxide nanotubes formed on the surface of titanium-containing foil or film are thought to be amorphous in nature. Proper annealing may form either of two crystalline structures that usually are found in titanium oxide crystals-the rutile and anatase crystalline phases. The annealing process preferably may be executed so as to select between the rutile and anatase phases of titanium oxide in accordance with a desired biological response. Annealing can also be used to decrease the electrical impedance |Z| of the nanotube array to less than 20 Ω·cm² at 1 kHz to enhance the suitability of the nanotube arrays for use in detecting neural signals without be overwhelmed by noise.

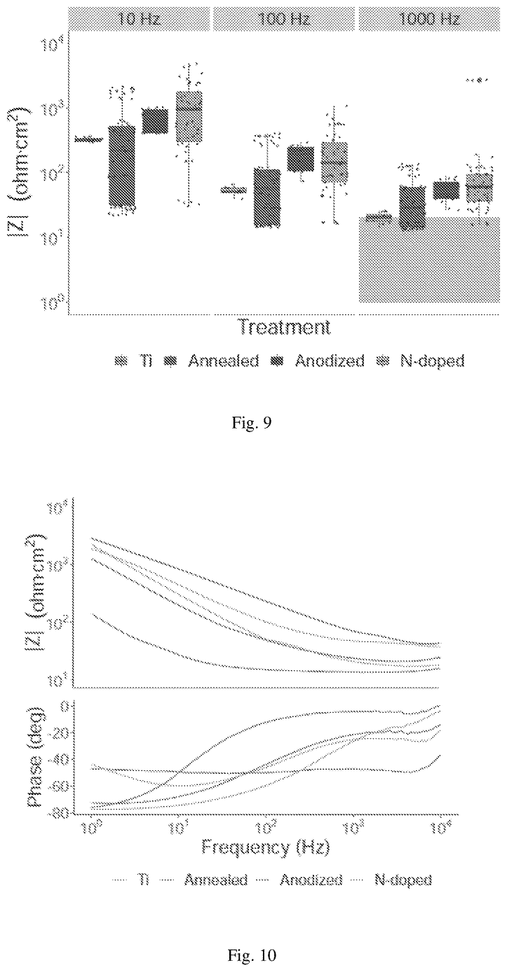

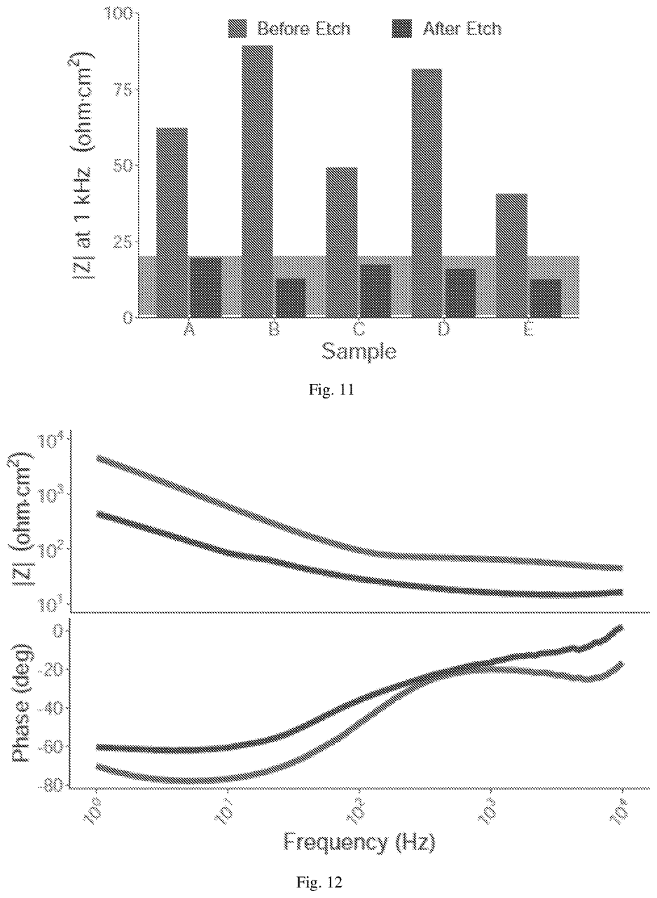

As shown in FIGS. 8-11, the electrical impedance of the nanotube arrays can be further enhanced by etching the annealed nanotube array to remove spongy oxide layer that can potentially form on a surface of the nanotube array as result of annealing. In some embodiments, the nanotube array can be etched by reactive ion etching in a CF₄O₂ plasma. Titania nanotube arrays annealed and etched can have an electrical impedance less than 20 Ω·cm² at 1 KHz.

In other embodiments, a coating can be provided on at least a portion of oriented nanotube array modify at least one property of the nanotube array. The coating can include, for example, a titanium nitride coating.

The oriented nanotube array can be loaded with at least one bioactive agent by adsorption, absorption, or a combination thereof. The bioactive agents may be adsorbed onto and incorporated into the nanotubes, by depositing a solution or dispersion containing the agents on the nanotubes, dipping the foil or film including the nanotubes into a solution or dispersion containing the agents, or by other means recognized by those skilled in the art. Optionally, the surface and pores of the nanotubes can be activated to promote covalent or non-covalent binding between the surface and the bioactive agent.

The oriented nanotube array can be configured modified or configured to differentially, controllably, spatially, and/or temporally release at least one bioactive agent the bioactive agent release coating. The nanotube arrays geometrical characteristics (e.g., nanotube pore diameter and length) strongly influence release kinetics, which can be tuned to sustain the release of the bioactive agent over several weeks or months. Accordingly, the bioactive agent release coating can provide sustained release of the bioactive agent upon implantation of the implantable medical device in a subject, preferably sustained release over a duration of time of weeks to months upon implantation.

In other embodiments, the nanotube pore diameter and length can be configured to facilitate release of one or more bioactive agents according to a specific temporal release profile. Alternatively, one or more materials or agents can be added to the nanotube array to facilitate differential and/or controlled release of one or more bioactive agents according to a temporal release profile.

In some embodiments, the nanotubes will release the absorbed or adsorbed bioactive agents in a time-controlled fashion. In this way, the therapeutic advantages imparted by the addition of bioactive agents and additives may be continued for an extended period of time. It may be desirable to include certain additives in the electrolyte solution used during the electrochemical anodization process in order to increase the adsorptive properties of the nanotubes of the device. For example, the inclusion of salts in the electrolyte solution used during the electrochemical anodization process may result in the incorporation of ionic substances into the nanotubes. The inclusion of ionic substances in the nanotubes may impart greater adsorptive properties to the nanotubes due to the polar interactions between the nanotubes containing ionic substances and the bioactive agents and additives.

The bioactive agents or additives loaded onto or in the nanotubes may be in a purified form, partially purified form, recombinant form, or any other form appropriate for inclusion in the nanotube array. It is preferred that the agents or additives be free of impurities and contaminants.

For example, growth factors may be included in the nanotube array to encourage bone or tissue growth. Non-limiting examples of growth factors that may be included are platelet derived growth factor (PDGF), transforming growth factor b (TGF-b), insulin-related growth factor-I (IGF-I), insulin-related growth factor-II (IGF-II), fibroblast growth factor (FGF), beta-2-microglobulin (BDGF II), and bone morphogenetic factors. Bone morphogenetic factors are growth factors whose activity is specific to bone tissue including, but not limited to, proteins of demineralized bone, demineralized bone matrix (DBM), and in particular bone protein (BP) or bone morphogenetic protein (BMP). Osteoinductive factors such as fibronectin (FN), osteonectin (ON), endothelial cell growth factor (ECGF), cementum attachment extracts (CAE), ketanserin, human growth hormone (HGH), animal growth hormones, epidermal growth factor (EGF), interleukin-1 (IL-I), human alpha thrombin, transforming growth factor (TGF-beta), insulin-like growth factor (IGF-I), platelet derived growth factors (PDGF), and fibroblast growth factors (FGF, bFGF, etc.) also may be included in the nanotube array.

Still other examples of bioactive agents and additives that may be loaded onto or in the nanotube array are biocidal/biostatic sugars, such as dextran and glucose; peptides; nucleic acid and amino acid sequences such as leptin antagonists, leptin receptor antagonists, and antisense leptin nucleic acids; vitamins; inorganic elements; co-factors for protein synthesis; hormones; endocrine tissue or tissue fragments; synthesizers; enzymes such as collagenase, peptidases, and oxidases; polymer cell scaffolds with parenchymal cells; angiogenic agents; antigenic agents; cytoskeletal agents; cartilage fragments; living cells such as chondrocytes, bone marrow cells, mesenchymal stem cells, natural extracts, genetically engineered living cells, or otherwise modified living cells; autogenous tissues such as blood, serum, soft tissue, and bone marrow; bioadhesives; periodontal ligament chemotactic factor (PDLGF); somatotropin; bone digestors; antitumor agents and chemotherapeutics such as cis-platinum, ifosfamide, methotrexate, and doxorubicin hydrochloride; immuno-suppressants; permeation enhancers such as fatty acid esters including laureate, myristate, and stearate monoesters of polyethylene glycol; bisphosphonates such as alendronate, clodronate, etidronate, ibandronate, (3-amino-1-hydroxypropylidene)-1,1-bisphosphonate (APD), dichloromethylene bisphosphonate, aminobisphosphonatezolendronate, and pamidronate; pain killers and anti-inflammatoirech as non-steroidal anti-inflammatory drugs (NSAID) like ketorolac tromethamine, lidocaine hydrochloride, bipivacaine hydrochloride, and ibuprofen; antibiotics and antiretroviral drugs such as tetracycline, vancomycin, cephalosporin, erythromycin, bacitracin, neomycin, penicillin, polymycin B, biomycin, Chloromycetin, streptomycin, cefazolin, ampicillin, azactam, tobramycin, clindamycin, gentamicin, and aminoglycocides such as tobramycin and gentamicin; and salts such as strontium salt, fluoride salt, magnesium salt, and sodium salt

In some embodiments, the bioactive agents loaded onto and released from the nanotube array can include one or more therapeutic agents that can be beneficial for promoting nerve regeneration and growth, reducing inflammatory responses to implantation of a neural interface, and/or minimizing scar tissue formation. Examples of such agents include a neurotrophin (e.g., nerve growth factor (NGF), neurotrophin-3 (NT-3), neurotrophin-4/5 (NT-4/5), a neuregulin (e.g., neuregulin 1, 2, 3, or 4), brain derived neurotrophic factor (BDNF), glial derived neurotrophic factor (GDNF), ciliary neurotrophic factor (CNTF), neurturin, artemin, persephin, glia maturation factor (GMF), or pituitary adenylate cyclase-activating polypeptide (PACAP)); a growth factor (e.g., CNTF, fibroblast growth factors (acidic and basic, aFGF and bFGF), transforming growth factor β (TGF-β), transforming growth factor α (TGF-α), GDNF, neurturin, epidermal growth factor (EGF), insulin-like growth factor (IGF), leukemia inhibitory factor (LIF), bone morphogenetic protein (BMP), or platelet-derived growth factor (PDGF)); a cytokine (e.g., an interleukin (IL), such as IL-1, IL-18, IL-2, IL-4, IL-10, IL-13, IL-6, IL-17, or IL-12; a tumor necrosis factor (TNF), such as TNF (formerly TNF-α) and lymphotoxin-alpha (formerly TNF-β); an interferon (IFN), such as IFN-α, IFN-α2a, IFN-α2b, IFN-β, IFN-β1α, IFN-γ, IFN-γ1b, and human leukocyte interferon-alpha (HuIFN-α-Le), including peglylated forms thereof); transforming growth factor β (TGF-β); erythropoietin (EPO); thrombopoietin (TPO); stem cell factor (SCF), a colony-stimulating factor (CSF, such as CSF1, CSF2, CSF3, or promegapoietin), or secreted phosphoprotein 1(SPP1 )); a chemokine, such as a CC chemokine (e.g., CCL1, monocyte chemoattractant protein-1 (CCL2/MCP-1), macrophage inflammatory protein-1α (CCL3/MIP-1α), CCL3L1, CCL3L3, macrophage inflammatory protein-1β (CCL4/MIP-1β), CCL4L1, CCL4L2, CCL5/RANTES, CCL6, CCL7, CCL8, macrophage inflammatory protein-1γ (CCL9/CCL10/MIP-1γ), eotaxin (CCL11), CCL12, CCL13, CCL14, CCL15, CCL16, CCL17, CCL18, CCL19, CCL20, CCL21, CCL22, CCL23, CCL24, CCL25, CCL26, CCL27, or CCL28), a CXC chemokine (e.g., CXCL1/KC, CXCL2, CXCL3, CXCL4, CXCL4L1, CXCL5,CXCL6, CXCL7, CXCL8, CXCL9, CXCL10, CXCL11, CXCL12, CXCL13, CXCL14, CXCL15, CXCL16, CXCL17, or LIX), a C chemokine (e.g., lymphotactin-α (XCL1) and lymphotactin-p/SCM-1 beta (XCL2)), or a CX.sub.3C chemokine (e.g., fractalkine (CX.sub.3CL1)); a lymphokine, e.g., IL-2, IL-3, IL-4, IL-5, IL-6, granulocyte-macrophage colony-stimulating factor (GM-CSF), or IFN-γ; a cell (e.g., a glial cell, a Schwann cell, a neuronal cell, a neural progenitor cell, a stem cell, a bone mesenchymal stromal cell (BMSC), an immune response cell (e.g., a lymphocyte such as a T-cell or a B-cell, a macrophage, a phagocyte such as a monocyte or a macrophage or a dendritic cell, a mast cell, a platelet, a white blood cell (e.g., including a neutrophil, a basophil, an eosinophil, a lymphocyte, a natural killer cell, and a monocyte), a red blood cell, an antigen presenting cell), etc.); a protein (e.g., an antibody or an enzyme such as chondroitinase ABC or a protease); a peptide (e.g., an affinity peptide, such as RGD, IKVAV (SEQ ID NO: 1), or YIGSR (SEQ ID NO: 2)); a drug, such as a steroid or an anti-inflammatory agent (e.g., dexamethasone or α-melanocyte stimulating hormone (α-MSH)); an axonal guidance protein (e.g., a netrin, a slit, a semaphorin, an ephrin, or a cell adhesion molecule (CAM)); an extracellular matrix (ECM) molecule (e.g., laminin, fibronectin, tenascin, a proteoglycan (e.g., heparan sulfate, chondroitin sulfate, or keratan sulfate), a polysaccharide (e.g., hyaluronic acid), a fiber (e.g., elastin or collagen, including fibrillar (Types I-III, V, and XI), facit (Types IX, XII, and XIV), short chain (Types VIII and X), basement membrane (Type IV), or other forms (Types VI, VII, and XIII), as well as other polymers including RGD, IKVAV (SEQ ID NO: 1), or YIGSR (SEQ ID NO: 2) binding sites); and/or a morphogen (e.g., Wnt or Sonic Hedgehog (SHH).

For any methods and devices described herein, single or multiple agents can be loaded on or into the nanotubes. In some embodiments, each agent can be released to have a unique release profile (i.e., temporal control). In other embodiments, each agent can be released in a manner that varies with the location of the agent (i.e., spatial control), where the location can be either within the z-dimension of the implant (e.g., along the thickness of the implant) or the x- or y-dimension of the implant (e.g., along the surface of implant).

For any of the approaches described herein, a skilled artisan would understand how to optimize the fabrication steps to promote particular release characteristics of the bioactive agent. For instance, the release profile of the bioactive agent can be further optimized by fine-tuning the pore size and length of nanotubes, the loading amount of one or more bioactive agents, the affinity of a coating to the bioactive agent, etc. In addition, release profiles can be controlled through selection of materials and methods of inclusion of the agent.

In some embodiments, the implantable medical device can include at least one nanotube array that can be processed in such a manner as to produce a functionally graded nanotube surface. For example, the metal foil or film may be partially immersed in the electrolyte solution during processing so that only a portion of the metal surface is processed to form nanotubes thereon. Alternatively, the implant may be gradually immersed or withdrawn from the electrolyte solution during processing so that more developed or taller nanotubes are formed on a portion of the implant's metal surface. For example, if the metal foil or film is immersed or removed from the electrolyte in a length-wise fashion, a graded nanotube surface spanning the portion of the length of the metal foil or film contacted by the electrolyte solution may be formed.

In other embodiments, a photoresist can be applied to select portions of the metal foil or film such that the metal foil or deposited metal film includes photoresist covered portions and uncovered portions. Providing select portions of the metal foil or film with the patterned photoresist allows only oriented nanotubes to form on uncovered portions of the valve metal foil or film during electrochemical anodization In some embodiments, the implantable medical device can include a transdermal implant with a biocompatible flexible substrate and a bioactive agent release coating that are configured to interface with epithelial tissue. The bioactive agent release coating can include vertically-oriented nanotube arrays of inorganic materials that can provide controlled release of bioactive agents, such as therapeutic agents, over an extended period of time to epithelial tissue. The transdermal implant can be used for a variety of applications in the form of general or stimuli-responsive skin patches for the treatment of chronic and/or non-healing wounds while preventing infections.

In other embodiments, the implantable medical device can be an implantable electrode assembly. The implantable electrode assembly can include a biocompatible flexible substrate and bioactive agent releasing microelectrodes that are configured to interface with tissue, such as neural tissue. The bioactive agent releasing microelectrodes can provide electrical stimulation to and electrical recording of neural tissue. The bioactive agent releasing microelectrodes can include inorganic nanotube arrays that are configured for bioactive agent, e.g., anti-inflammatory, antioxidant, growth factor, genes, etc., release with (high spatial precision) with simultaneous electrical recording and stimulation.

The implantable electrode assembly can be used in several applications including, but not limited to, flexible electronics substrates for use in central nervous system neural interfaces and peripheral nervous system neural interfaces. For example, an implantable electrode assembly used in a central nervous system neural interface can include an intracortical electrode array (motor cortex, somatosensory cortex, auditory cortex, visual cortex), inferior colliculus implant, deep brain stimulator, electrocorticography array, spinal cord electrode and brain stem implant. An implantable electrode assembly used in a peripheral nervous system neural interface can include nerve cuff electrodes, vagus nerve electrodes, dorsal root ganglion electrodes, intrafascicular electrodes, regenerative electrode arrays, electromyography arrays, cochlear electrodes. Additionally, the implantable electrode assembly described herein can be used in devices that interface with the cardiovascular system, nervous system, lymphatic system, pulmonary system, endocrine system, musculoskeletal system.

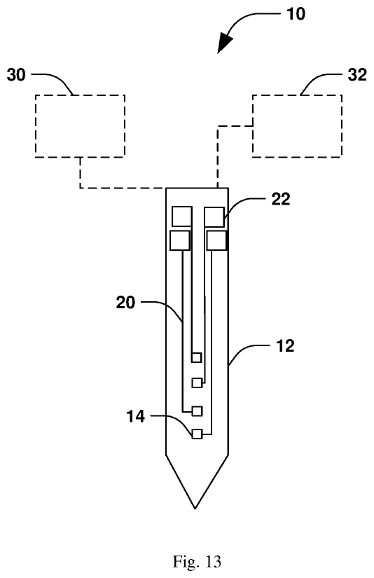

FIG. 13 illustrates an example of a flexible electrode assembly 10 that can be used as neural interface for neural stimulation and recording. The flexible electrode assembly 10 allows the user the ability to study the functions of neurons and their associated networks within a subject. This can include the study of the function of neurons located within a particular volume or area of interest as well as the study of functions of neurons located outside of the particular volume or area. The assembly provides for the capture of neural signals of the subject and/or injection of a signal into a neuron or neural system of the subject.

The flexible electrode assembly 10 includes an elongated flexible electrode shaft 12 and a plurality of bioactive agent releasing microelectrodes 14 arranged on the flexible electrode shaft 12 that are configured to interface with tissue, such as neural tissue. Although only one flexible electrode shaft 12 is illustrated in FIG. 13, the flexible electrode assembly 10 can include multiple flexible electrode shafts arranged in a three dimensional array. The flexible electrode shaft can have a shape that makes it suitable for implantation in neural tissue, such as intracortical implantation in the brain. To make it suitable for implantation, the electrode shaft 12 may be manufactured of a tissue-biocompatible flexible polymer, such as a polyimide or SU8. The skilled person will however appreciate that other biocompatible flexible insulating materials in combination with electrically conductive materials may also be suitable.

The length of the electrode shaft 12 can be about 2 mm to about 40 mm, for example, about 5 mm 40 mm, about 5 mm to about 30 mm, or about 7 to about 25 mm, so that electrode shaft can extend beyond a single neuron, or cover even plural folded gyrus in the cerebral cortex.

The microelectrodes 14 arranged on the electrode shaft 12 are distributed in such a manner over the shaft 12 that they are able to make electrical contact with a region of the neural tissue, e.g., cerebral cortex. Each microelectrode 14 includes an oriented nanotube array, such as a titania nanotube array, that is loaded with a bioactive agent that can be beneficial for promoting nerve regeneration and growth, reducing inflammatory responses to implantation of a neural interface, and/or minimizing scar tissue formation. The nanotubes of the nanotube array are oriented in substantially the same direction relative to the surface of the flexible electrode shaft 12. For example, the nanotubes of the nanotube array can be oriented substantially perpendicular to the surface of the flexible shaft.

The nanotubes of the oriented nanotube array can have an average and/or mean pore diameter in the range of about 1 to about 100 nm (e.g., about 5 nm, about 10 nm, about 20 nm, about 30 nm, about 40 nm, about 50 nm, about 60 nm, about 70 nm, about 80 nm, about 90 nm, about 100 nm, about 125 nm, about 150 nm, about 200 nm) and an average and/or mean height in the range of about 0.5 μm to about 30 μm (e.g., about 0.5 μm, about 1 μm, about 2 μm, about 3 μm, about 4 μm, about 5 μm, about 6 μm, about 7 μm, about 8 μm, about 9 μm, about 10 μm, about 12 μm, about 15 μm, or about 20 μm, about 22.5 μm, about 25 μm, about 27.5 μm, about 30 μm).

In some embodiments, the nanotubes are annealed and/or reactive ion etched so that each microelectrode 14 has a low electrode-electrolyte impedance. For example, each microelectrode can have an electrical impedance |Z|<20 Ω·cm² at 1 kHz.

Each microelectrode 14 comprising a nanotube array can have a diameter of, for example, about 10 μm to about 200 μm, or about 50 μm to about 100 μm. While the microelectrodes 14 are illustrated as being rectangular, the microelectrodes can have other shapes including, for example, a circular or annular shape. The oriented nanotube array of the microelectrode 14 can be modified or configured to differentially, controllably, spatially, and/or temporally release at least one bioactive agent the microelectrode. The nanotube arrays geometrical characteristics (e.g., nanotube pore diameter and length) strongly influence release kinetics, which can be tuned to sustain the release of the bioactive agent over several weeks or months. Accordingly, the microelectrode 14 can provide sustained release of the bioactive agent upon implantation of the flexible electrode assembly in a subject, preferably sustained release over a duration of time of weeks to months upon implantation.

Each microelectrode 14 arranged on the flexible electrode shaft 12 can be controlled individually such that some of the microelectrodes 14 of a single electrode shaft 12 are electrically driven or activated by applying a stimulation signal, and others are not operated. Each microelectrode may also be operated for either signal stimulation or signal recording. In an operational mode, the microelectrodes are operated for signal stimulation only. In a configuration mode, the microelectrodes may however also be controlled to operate in a signal recording modus such that neural activity or neural response to the stimulation signal may be recorded accordingly. In an example, each microelectrode may be arranged such that it can operate both in the stimulation and recording modus, and in an alternative example, microelectrodes may be dedicated for either signal stimulation or signal recording.

In order to process the signal for neural stimulation or neural recording, the microelectrodes 14 of the flexible electrode assembly 10 can be electrically connected via electrical interconnects 20 and contact pads 22 to a driving unit 30 and a recording unit 32. These units may be contained in separate housings, at separate locations in or on the patient, but are preferably contained in a transducer device (not shown) which may be close to or integrated with the shaft or positioned directly under the scalp on the skull, for example in or near the layer of loose connective tissue of the scalp.

Optionally, the flexible electrode shaft 12 can also include a rigid electrode support structure (not shown) to allow penetration of the surface tissue of the cerebral cortex. The support structure can include a rigid or stiff insert shafts. The flexible electrode shaft can be attached or fixed to the insert shaft of the support structure upon intracortical implantation in the cerebral cortex. Once inserted, the insert shaft disengages such that the insert shaft can be retracted, whereas the electrode shaft remain in place in the cerebral cortex. Attaching and disengaging can be achieved in different ways. For example, the insert and respective electrode assembly may be temporarily attached by a brain fluid dissolvable adhesive such as PEG. This example may well work with flat ribbon shaped electrodes.

The electronics of the driving unit 30, to electrically drive the stimulation of the subset of microelectrode 14 locations, as well as the recording unit 32, to obtain neural recording through the microelectrodes 14 in the region of the cerebral cortex, can be packaged inside the implantable transducer casing and shielded from the tissue by the material of the casing, which is preferably made of titanium. In case the electronics or electronic circuitry may reside on the flexible electrode assembly 10 or more particular, they can be embedded in a tissue compatible packaging material.

The flexible electrode assembly can also include of a switching unit (not shown). The switching unit consists of electronic circuitry which channels stimulation signals from the driving unit 30 to the microelectrodes 14 located in the cerebral cortex. The driving unit 30 may be equipped with circuitry to generate a plural stimulation signal, i.e., a stimulation pulse of a particular waveform such as a sine wave form, a square wave form, a rectangular wave form, a triangular wave form, a sawtooth wave form, a pulse wave form or any combination of wave forms. The generated signals may not only differ in shape but also in amplitude and can be channeled to different individual or subsets of microelectrodes. This way some groups or subsets of microelectrodes may not be activated at all, others with a first stimulation signal, yet another group with a second, different stimulation signal, and so on. To this end, the switching unit is arranged to achieve electrical channeling of the different signal generators and the subsets of electrical contacts.

To control the driving unit 30, the recording unit 32, and thereby the electrodes 14 of the electrode assembly a processing unit (not shown) can be provided. The processing unit can be a general-purpose hand-held device, such as the mobile phone, or a dedicated device with dimensions that are preferably similar to those of a mobile phone.

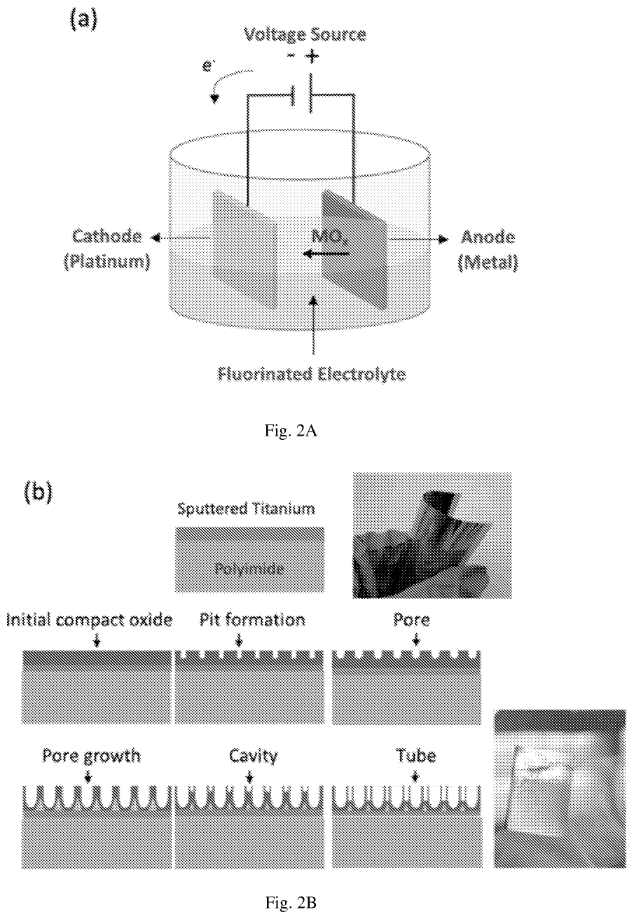

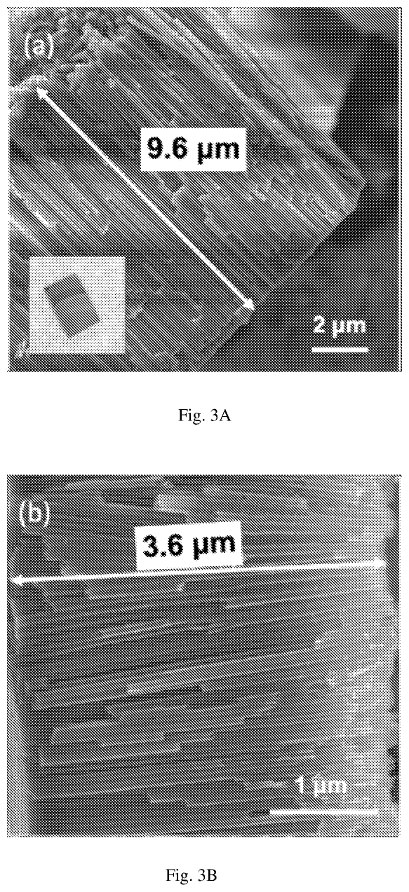

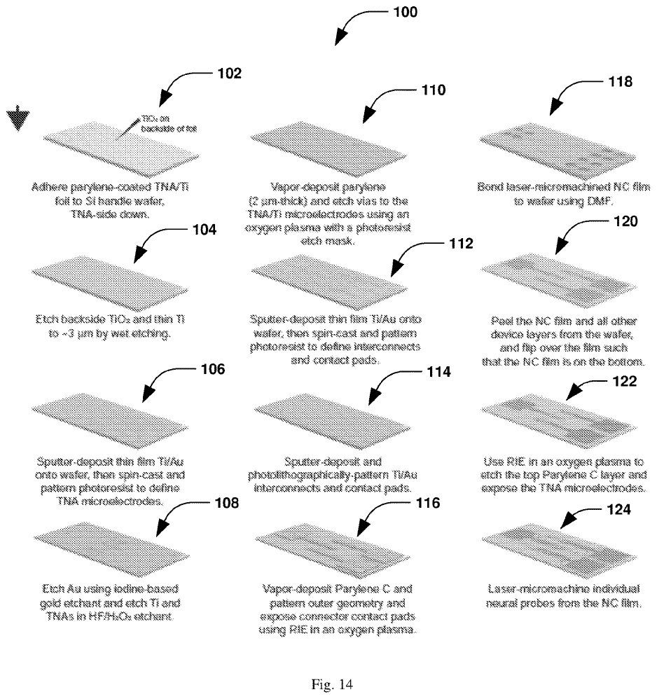

FIG. 14 is a schematic illustration of an example of a method 100 forming a flexible electrode assembly in accordance with an embodiment described herein. In the method, at step 102, a titania nanotube array is initially formed by on a thin titanium foil (e.g., about 25 μm) by electrochemical anodization of the titanium foil in a fluorinated electrolyte (e.g., 0.5 wt. % of ammonium fluoride (NH₄F), 3 vol. % of deionized water, and 96 vol. % ethylene glycol) as shown in FIG. 2A-2B. The electrochemically anodized titanium foil includes a first surface with a titania nanotube array and opposite second surface that is devoid of titania nanotubes. The first surface including the titania nanotube array is coated with parylene to protect the titania nanotube array, and then then first surface of the titanium foil is adhered to a silicon wafer so that opposite second surface exposed for further processing. At step 104, the exposed opposite second surface is then etched by, for example, wet etching to reduce the thickness of the titania foil to about 3 μm.

Following etching of second surface of the titanium foil, at step 106, gold is sputter deposited on the second surface of the titanium foil to provide a thin gold layer overlying the second surface of the titanium foil. A photoresist is spin-cast and patterned on the deposited gold layer such that the patterned photoresist overlies the gold layer and titanium in positions that define the dimensions or areas of portions of the titania nanotube array that form the microelectrodes.

At step 108, the gold layer is then etched using, for example an iodine gold etchant, and the underlying titanium foil and titania nanotube array are further etched, using a HF/H₂0₂ etchant to remove portions gold and titanium foil not covered by or not underlying the patterned photoresist resulting in a plurality microelectrodes that each include an underlying titania nanotube array and an overlying gold layer. The remaining patterned photoresist overlying the microelectrodes can then be removed.

At step 110, a parylene layer (e.g., about 2 μm) is then deposited over the microelectrodes and silicon wafer and the parylene layer is etched using oxygen plasma with a photoresist etch mask to provide vias to the microelectrodes underlying the parylene layer.

At step 112, a thin film of titanium/gold is deposited onto the wafer and a photoresist is spin cast and patterned to define Ti/Au interconnects and contact pads overlying the microelectrodes.

At step 114, the Ti/Au interconnects and contact pads are then sputter-deposited and photolithographically patterned.

At step 116, Parylene C is vapor deposited on interconnects and contacts, the outer geometry is patterned, and contact pads are exposed by reactive ion etching the parylene C using oxygen plasma.

At step 118, a flexible micromachined polymer nanocomposite (NC) film that includes a poly(vinyl acetate) matrix and a cellulose nanocrystal filler can then be bonded to the wafer using DMF so that the NC film overlies the microelectrodes, interconnects, and contact pads.

At step 120, the NC film and all other device layers including the microelectrodes, interconnects, and contact pads, can be peeled from the wafer and the peeled layers can be flipped so that the microelectrodes are exposed and overly the NC film.

At step 122, the parylene C layer covering the titania nanotube array of the microelectrodes can be etched by reaction ion etching to expose the titania nanotube array of the microelectrodes.

At step 124, the NC film can be micromachined to provide flexible electrode assemblies that include the plurality of microelectrodes.

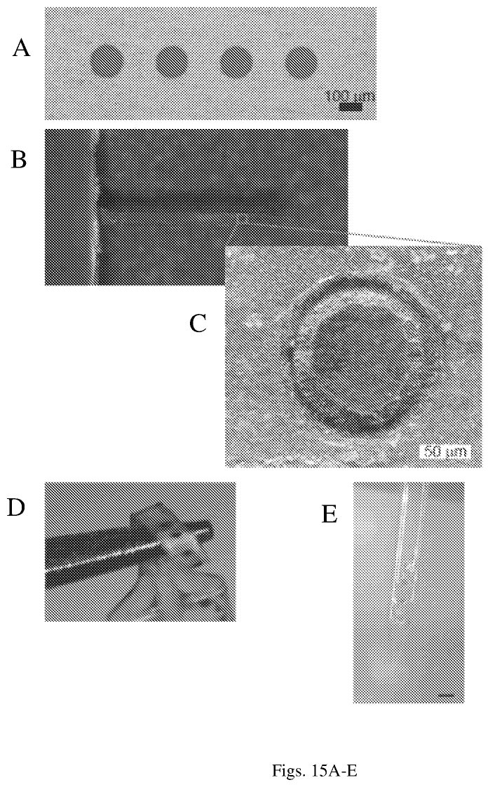

FIG. 15(A-E) show (A) TNA microsegments patterned on PDMS film. (B) Completed TNA-NC neural probe connected to printed circuit board. (C) Individual TNA microelectrode integrated into TNA-NC neural probe with Au trace. (D) TNA-NC neural probe wrapped around tweezers to demonstrate mechanical flexibility. (E) TNA-NC neural probe inserted into agarose gel demonstrates functional robustness of the TNAs integrated onto the mechanically-softening substrate with interconnecting traces. Scale bar =200 μm

The following examples are included to demonstrate various embodiments of the invention and are not intended to be a detailed catalog of all the different ways in which the present invention may be implemented or of all the features that may be added to the present invention. Persons skilled in the art will appreciate that numerous variations and additions to the various embodiments may be made without departing from the present invention. Hence, the following descriptions are intended to illustrate some particular embodiments of the invention, and not to exhaustively specify all permutations, combinations and variations thereof

### Example

In this example, we show the development and integration of titania nanotube arrays (TNAs) on flexible substrates as a multifunctional platform to interface with soft tissue (e.g., nervous system). We demonstrate different approaches for the incorporation of TNAs with two different polymer substrates using electrochemical anodization and microfabrication techniques. By integrating the TNAs and the polymer films, we generated a device that maintains the drug eluting and biocompatible properties of the TNAs with the flexibility of the polymeric substrate.

### Materials and Methods

### Titanium Deposition on Polyimide

Titanium films (˜0.3 μm-1 μm) were deposited on Dupont 300HN Kapton (polyimide, (2 mil thick) substrates using the DC magnetron sputtering technique (Denton Vacuum Discovery 18, USA). Kapton sheet was cut into 4 cm×2 cm size pieces and the pieces were ultrasonically cleaned with deionized (DI) water, ethanol, and isopropyl alcohol (IPA) and finally dried using nitrogen gun. The Kapton pieces then were loaded into the sputtering chamber (Denton Vacuum Discovery 18). The base pressure was 8.4×10⁻⁷ Torr, and the power was 250 W. The film deposition process was conducted at 3 m Torr argon pressure, with a 75 mm distance between the cathode and substrate.

### Fabrication and Characterization of Tnas

The electrochemical anodization of Ti foils and sputtered Ti on Kapton substrates was performed in a mixture of 0.5 wt % of ammonium fluoride (NH₄F), 3 vol % of deionized (D.I.) water and 96 vol % of ethylene glycol (EG). High purity polished titanium foils (25 μm thick, 99.98%, Sigma-Aldrich) were sonicated in deionized (D.I.) water, ethanol and isopropyl alcohol (IPA), separately, and then dried in air. The anodization was performed in a two-electrode set-up with Ti as the anode and the platinum foil (99.99%, Sigma-Aldrich) as the cathode at different voltages ranging from 10 V to 40 V. The surface morphology of the films was observed using an Inspect F50 scanning electron microscope.

### Integration of Tnas With Polymer Substrates

TNA microsegments were fabricated on a polymer substrate using polydimethylsiloxane (PDMS, Sylgard 184, Dow Corning) that was mixed in a 10:1 ratio, degassed and spin-cast to a thickness of 50 μm onto a silicon wafer. The PDMS was partially cured for 15 minutes at 70° C. Anodized (40V/1.5 h) TNA/Ti foils were then placed TNA-side down onto the partially-cured, tacky PDMS. Subsequently, the wafer was placed in an oven (1 hour, 70 C) to fully cure the PDMS. At this point, the TNA/Ti foils were securely attached to the PDMS to facilitate further processing. Next, the TNA/Ti foils were thinned to approximately 6 μm by etching the Ti in 5% HF for 5 minutes. Using an AZ nLOF 2070 photoresist etch mask, then foil was then etched in a 1% HF solution for approximately 10 minutes to remove all titanium and nanotubes between the defined microsegments. After rinsing and drying, the photoresist was removed using acetone and isopropanol. At this point, patterned TNA microsegments were adhered to the PDMS coating the silicon wafer.

To demonstrate the process, SU-8 (SU-8 2015) was spin-cast onto the wafer to a thickness of 20 μm. The SU-8 was soft-baked, flood exposed with UV light, then post-exposure baked.

The SU-8 and the embedded TNA microsegments were then transferred to the SU-8 substrate.

Ti foil (˜3″ in by 3″) was lightly adhered to a silicon wafer using water. The silicon wafer and titanium foil were then placed in a spin coater and ˜5 mL of AZ nLOF 2070 was applied to the surface of the titanium foil. Next the sample was soft baked on a hotplate for 90 seconds at 110° C. prior to exposure to UV light through a photomask. A post exposure bake was additionally conducted at 110° C. for 90 seconds. The sample was then developed utilizing 300 MIF photoresist developer via immersion for 2 minutes before being rinsed in DI water and dried using a nitrogen gun. Filmetrics thin film analyzer was used to identify the surface thickness of the photoresist. The patterned photoresist-coated titanium foil was cut into ˜0.5″×0.5″ segments and removed from the silicon wafer. The sample was then hard baked on a hot plate for 2 minutes at 140° C. After hard baking, the sample was subjected to the standard anodization procedure at 40 V for 5 hours. After anodization, the sample was removed from the anodization solution, rinsed in DI water, and dried using a nitrogen gun. The photoresist mask was then removed using reactive ion etching (RIE) in an oxygen plasma at 150 W for four minutes.

### Drug Loading and Release Rate Measurements

Drug loading on samples was performed by micro-pipetting dexamethasone solution (25 μg/mL of dexamethasone sodium phosphate, molecular weight: 516.4 Da, Sigma Aldrich, dissolved in 1:1 ethanol and DI water) onto the surface of TNAs. A total of 500 μL of DEX solution was pipetted onto the nanotube surface (pipetting 100 μL for 5 times) and allowed to dry in fume hood for an hour, ensuring that the nanotube array surface was completely dry prior to each pipetting and the release studies. Then, DEX-loaded TNA samples were immersed in Eppendorf tubes containing 100 μL of PBS (1× phosphate buffered saline) and were placed in a water bath kept at 37° C. For release measurements, all 100 μL from each releasing sample tube was transferred to a 96-well plate and measured using a SpectraMax M 2 Microplate Reader. The absorption of DEX was measured at 244 nm. After each measurement, the releasing medium was fully replaced with 100 L of fresh PBS. Hourly measurements were taken for the first three hours of release, after which daily measurements were taken for 43 days. A standard curve with known concentrations of dexamethasone was used to determine the unknown concentrations of DEX.

### Results

### Controlled Diffusion and Linear Release Kinetics in Titania Nanotube Arrays

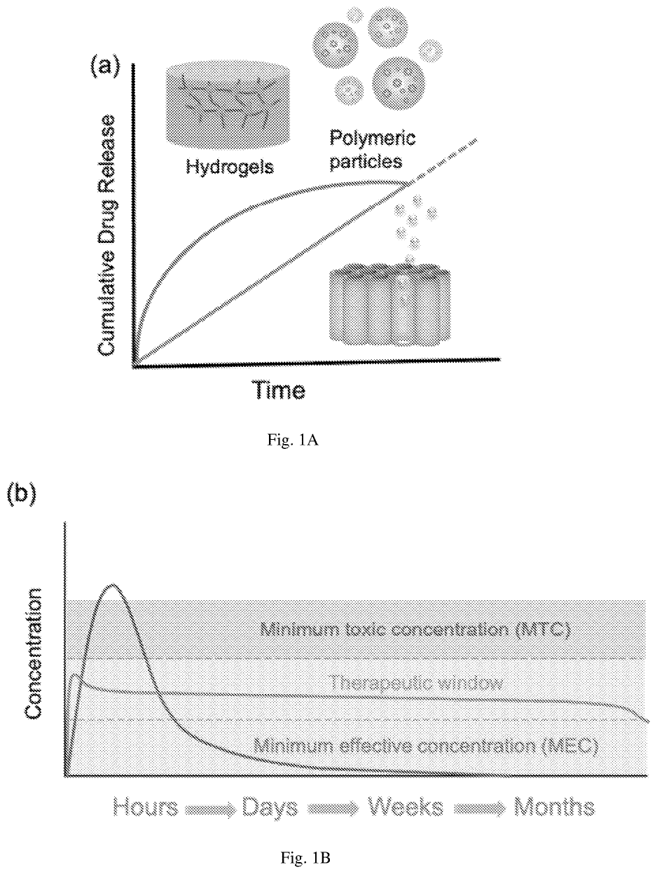

Swelling-controlled diffusion mechanisms underlie hydrogel-mediated drug release, where the extent of drug diffusion increases with swelling. Release profiles from hydrogels are typically characterized by an initial burst release, followed by a rapid decline in release rate, with complete depletion occurring within hours or days. In contrast, diffusion-driven drug release in nanopores is correlated with the relative size of drug molecule and nanotube pore (FIG. 1A). Under standard conditions, the diffusion rate is proportional to the concentration gradient, according to Fick's laws. When molecules are significantly smaller than the pore size, release follows a Fickian (first-order kinetics), nonlinear diffusion profile. When the pore size approaches the size of the molecule, the diffusion rate becomes pore size dependent-a phenomenon known as hindered or restricted diffusion which results in zero-order (linear) non-Fickian release profile (FIG. 1B). In this case, molecules diffuse through the nanotube pores at a linear rate that no longer correlates with the concentration gradient across the membrane. By tuning the nanotube geometry, controlled constant-rate delivery of a range of molecules with desired release profiles can be obtained. Accordingly, the zero-order release kinetics can overcome unfavorable burst release-characteristic of hydrogels, therefore, allowing for drug concentration near the implant to be sustained within the therapeutic window (FIG. 1C). However, interfacing TNAs with soft tissue/organ systems require a new platform to minimize tissue damage.

### Direct Growth and Patterning of Tnas on Polyimide Substrates

The formation of TNA is largely affected by the charge transfer properties of the titanium layer (or the substrate in contact with Ti). In contrast to pure metal substrates, anodizing metals on low-conductivity substrates is more complex. It is essential for the substrate to have sufficient electrical conductivity to allow the charges to pass through the substrate and distribute the electrical field along the thickness to the surface of titanium film. FIG. 2 shows the electrochemical anodization set up (FIG. 2A) and TiO₂ nanotube formation mechanism on titanium sputtered on polyimide substrate (FIG. 2B). By anodizing the thinner Ti-sputtered polyimide samples (Ti thickness ˜0.3 μm) at 40 V for 10 mins, TNAs with average pore size ˜85 nm, and the tube length ˜450 nm was achieved. The wall thickness of nanotube arrays on Kapton substrate is much thicker than tubes grown on pure Ti substrate. This could be due to the tapered shape of the nanotubes, where the walls are thicker at the bottom and thinner at the surface, as such the thicker walls resulted from shorter anodization time. Nanotubes become thinner near the surface as they grow longer, resulting in larger pores near their surfaces. The anodization of Ti-sputtered polyimide was controlled by monitoring anodization current profile over time. The formation of nanotubes in this case is very sensitive to the electrolyte composition, anodization time and post-cleaning; without these considerations, the nanotube layer can easily be dissolved or damaged.

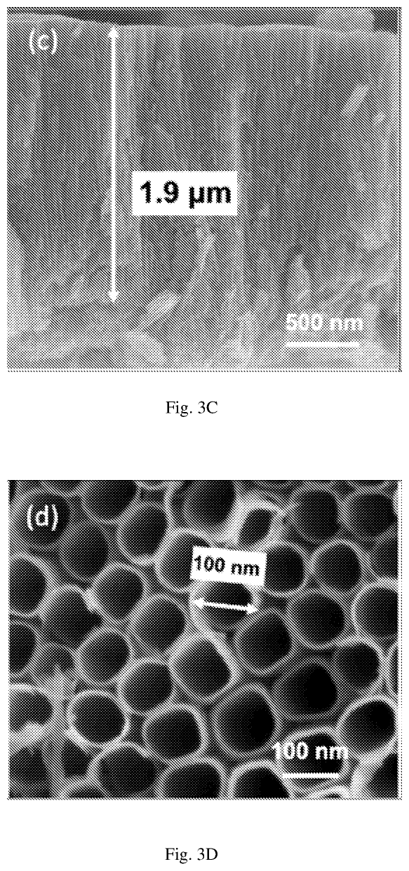

FIG. 3 shows the top and cross-sectional view SEM images of nanotubes anodized at 40 V, 20 V, and 10 V, respectively. The nanotubes anodized at higher voltage have larger pores (˜100 nm) and as the voltage decreases, pores become smaller (˜30 nm). Similar trends are observed in nanotubes length due to the slower dissolution rates as a result of lower voltage as a driving force for pore formation and growth.

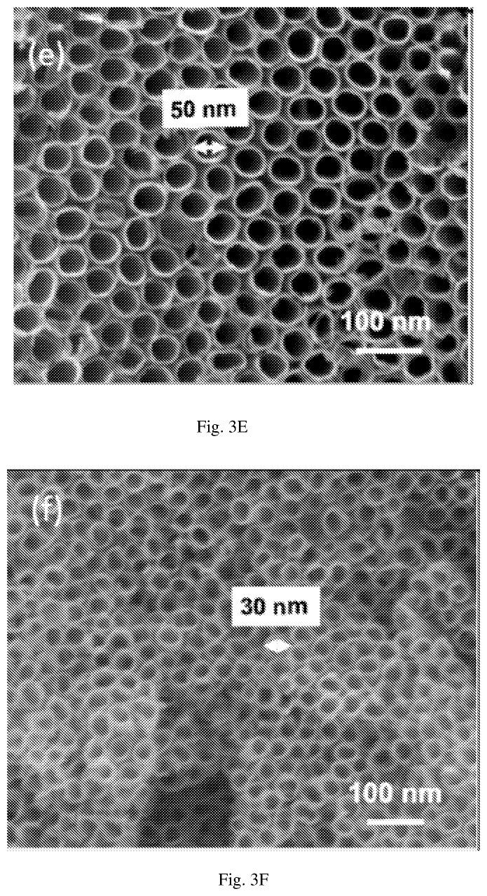

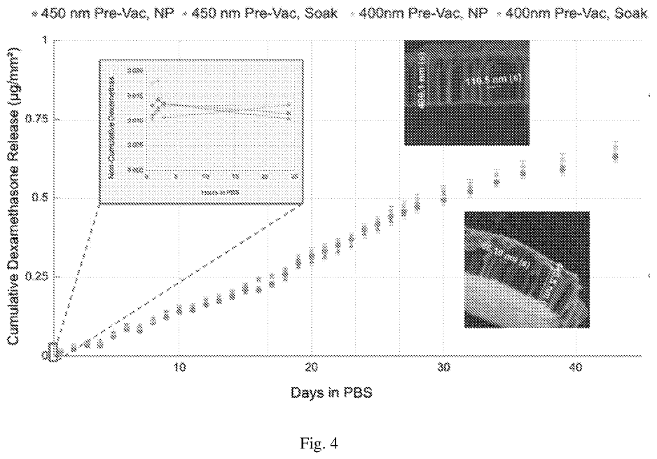

A linear release profile for an anti-inflammatory drug (Dexamethasone) was obtained at 37° C. from a ˜0.5 μm-deep, 100 nm-diameter TNAs on polyimide for at least 7 weeks (FIG. 4). No significant difference in release data was observed between the two drug loading methods (normal pipetting and soaking).

FIG. 5 shows an example of patterned TNAs on polyimide substrate using picosecond laser micromachining. The results revealed some color variation in the 30 μm-diameter microsegments on the left is due to thickness variation at the edges. This would arise due to the spatial spread of the laser beam —-this would give rise to some removal of material (in thickness direction) near to the cut.

### Patterning and Transferring TNA Films to Polymer Substrates

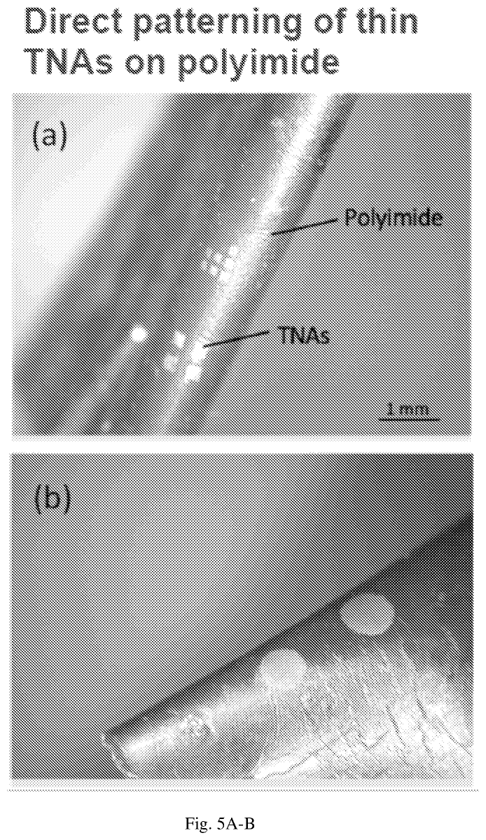

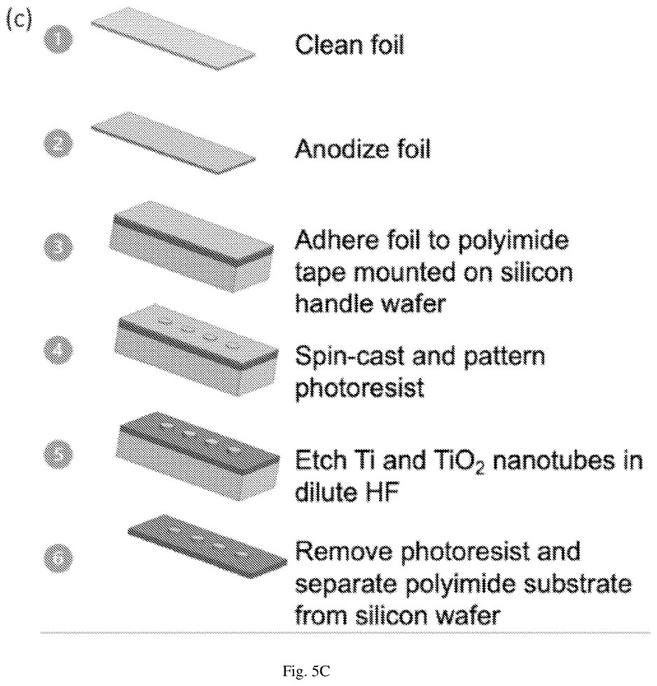

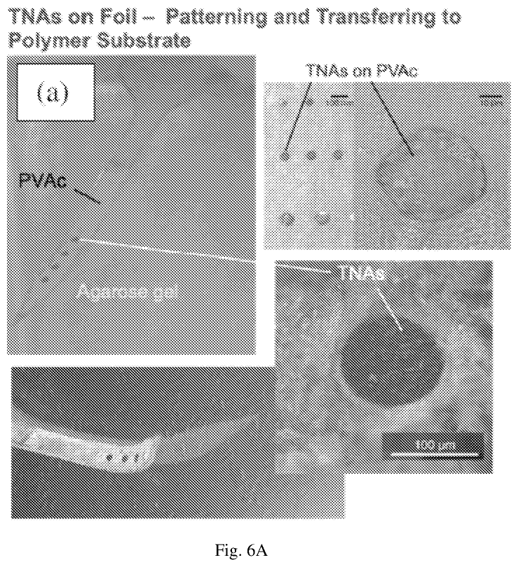

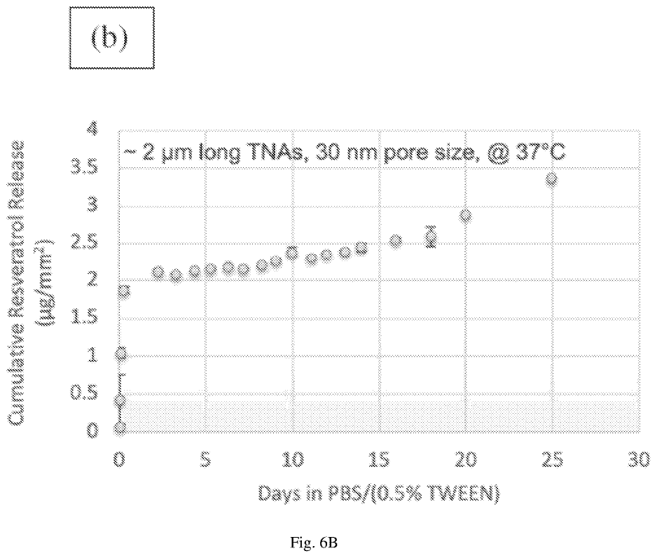

FIG. 6A shows a patterned device with different sizes of microsegments on an anodized foil and transferer to the flexible substrate. Patterning and transferring allows for performing all materials processing steps on the nanotubes samples before introducing the temperature-sensitive substrate materials. While the TNAs may be rigid and brittle, by patterning them into smaller areas on a flexible substrate, the device can still flex globally without adding too much stress to the TNAs. FIG. 6B Sustained release of antioxidant Resveratrol from the TNAs for nearly one month. Confirmation of surface feature quality and complete removal of AZ nLOF 2070 was confirmed using scanning electron microscopy (SEM).

We described methods of forming and integrating anodic TNAs on non-titanium flexible substrates in order to expand the applications of TNAs in implantable biomedical devices. One approach is to deposit a uniform layer of titanium on the substrate, followed by electrochemical anodization. Sputtering and evaporation techniques can produce high quality films with good adhesion; however, film thickness is limited to producing up to 2 μm thick films, which may be suitable for short-term drug delivery applications. Electrochemical anodization provides a relatively facile and scalable technique for the fabrication of porous inorganic materials, in particular, vertically-oriented nanotube arrays of various geometries from a selected group of materials (classified as valve metals). Uniform metal-oxide nanotube arrays with adjustable pore size, tube length and wall thickness can be fabricated by tailoring the electrochemical anodization parameters such as the applied voltage, reaction time, anodization temperature and composition of the electrolyte. The primary advantage of titania nanotube arrays is that the anodizing process is controllable and versatile, making it possible to produce nanotubes with precise dimensions based on the drug molecule size and the therapeutic requirements.

To utilize the TNAs for applications requiring sustained drug delivery, longer nanotubes (e.g., >2 μm) need to be developed and robustly integrated with various substrates. In doing so, we must also consider that TNAs are brittle materials, and therefore cannot flex much without being fractured unless they are very thin. Intrinsic stresses become more significant with increasing TNA thickness, which is particularly of concern on flexible substrates. However, TNAs can be patterned into microscale features integrated onto a flexible substrate, to provide both the benefits of long TNAs and the benefits of flexible substrates. To address this issue, and to generate longer nanotubes, high quality titanium foils of various grades and thicknesses can be anodized and integrated with polymer substrates.

The first approach involved the application and patterning of a photoresist to allow for the site-specific anodization and growth of TNAs on a titanium foil. By applying photoresist, we can allow specific segments of the titanium foil to be exposed to the anodization solution and thus manipulate the geometry and size of nanotubes. Moreover, we were interested in exploring if this method can be applied to other substrate materials that might not be chemically compatible with the anodization solution.

In the second approach, microsegments of TNA were encapsulated in polymer from both bottom and sidewalls to ensure a well-integrated stable structure for long-term implant applications. The photolithography, allowed for simultaneous patterning of numerous microsegments as small as 20 μm×20 μm. This approach is applicable to the sputtered films as well and allows for complex device designs. The most significant advantage of this method is that it enables high-temperatures post-anodization processes that are incompatible with polymer substrates or adhesives but can be useful for enhancing stability or other properties of TNAs, to be performed before integration process.

In vitro release rate studies were conducted to assess nanotube diameter affect release profiles. A linear release profile for an anti-inflammatory drug (Dexamethasone and Resveratrol) was obtained at 37° C. from TNAs on polyimide fand foil substrates for several weeks with no indication of a large burst after contacting the PBS. Release profiles obtained through in vitro studies on several different nanotube diameters within the 30 nm-100 nm range showed a weak relationship between nanotube diameter and DEX release rate, which we attribute to the relatively large difference in size between DEX molecules and the pore. TNAs can hold more than 10 times more drug than conductive polymers. Dexamethasone as an anti-inflammatory drug was used in this study as a model drug.

In summary, this example shows different approaches for integrating microscale TNA segments into polymer substrates that allows for spatially selective integration of long TNAs while maintaining the mechanical flexibility of the polymer. Sustained linear drug release was obtained from ˜0.5 μm-deep, 100 nm-diameter TNAs on polyimide for at least 7 weeks. These results make flexible TNAs a promising material platform for the development of multifunctional bio-interfaces.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. An implantable medical device comprising:
a biocompatible flexible substrate and a bioactive agent release coating on at least a portion of a surface of the substrate, the bioactive agent release coating comprising an oriented nanotube array of an inorganic material and at least one bioactive agent disposed on and/or in the nanotube array, the nanotube array providing delayed or extended release of the at least one bioactive agent from the coating.

2. The implantable medical device of claim 1, wherein the oriented nanotube array comprises a valve metal or valve metal oxide nanotube array.

3. The implantable medical device of claim 1, wherein the oriented nanotube array comprises an oriented nanotube array of an anodized valve metal.

4. The implantable medical device of claim 1, wherein the oriented nanotube array comprises a titanium or titania nanotube array.

5. The implantable medical device of claim 1, wherein the nanotubes of the nanotube array are oriented in substantially the same direction relative to the surface of the substrate or wherein the nanotubes of the nanotube array are oriented substantially perpendicular to the surface of the substrate.

6. (canceled)

7. The implantable medical device of claim 5, wherein nanotubes of the nanotube array have a pore diameter of about 30 nm to about 100 nm and a length of about 0.5 μm to about 30 μm.

8. The implantable medical device of claim 7, wherein the nanotubes are annealed and/or reactive ion etched.

9. The implantable medical device of claim 1, wherein the oriented nanotube array is configured to define at least one electrode on the surface of the substrate.

10. The implantable medical device of claim 9, wherein the oriented nanotube array has an electrical impedance |Z|<20 Ω·cm² at 1 KHz.

11. The implantable medical device of claim 7, wherein the nanotubes include at least one coating.

12. The implantable medical device of claim 11, wherein the at least one coating includes a titanium nitride coating.

13. The implantable medical device of claim 1, wherein the biocompatible flexible substrate comprises a compliant biocompatible polymer or textile.

14. The implantable medical device of claim 1, wherein the biocompatible flexible substrate comprises a polyimide film or sheet.

15. The implantable medical device of claim 1, wherein the bioactive agent release coating provides sustained release of the bioactive agent over a duration of time of weeks to months upon implantation of the medical device in a subject.

16. The implantable medical device of claim 1, wherein the device is a transdermal implant.

17. (canceled)

18. An implantable electrode assembly comprising the implantable medical device of claim 1.

19. The implantable electrode assembly of claim 18, wherein the oriented nanotube array defines at least one electrode that extends outward from the substrate and is configured to interface with neural tissue of a subject.

20. The implantable electrode assembly of claim 19, further comprising at least one electrical interconnect that electrically connects the at least one electrode to a driving unit and/or recording unit, the driving unit driving the electrode for electrically stimulating regions of the neural tissue and the recording unit for obtaining neural recording from the electrode.

21. The implantable electrode assembly of claim 20, wherein the electrode assembly is an intracortical electrode array, an inferior colliculus implant, a deep brain stimulator, an electrocorticography array, a spinal cord electrode, or a brain stem implant.

22. The implantable electrode of claim 21, wherein the bioactive agent released by the oriented nanotube array comprises at least one of a neuromodulation agent or an anti-inflammatory agent.

23-46. (canceled)
